(19) United States
(12) Patent Application Publication
(10) Pub. No.: US 2004/0171069 A1
(43) Pub. Date: Sep. 2, 2004

(54) Kinetic assay

(76) Inventors: Christian John Cook, Baglan (NZ); Vinqiu Wu, Hamilton (NZ); John Stanton Mitchell, Botobua (NZ)

Correspondence Address: JACOBSON HOLMAN PLLC, 400 SEVENTH STREET N.W., SUITE 600, WASHINGTON, DC, 20004, US

(21) Appl. No.: 10/477,191

(22) PCT Filed: May 14, 2002
(86) PCT No.: PCT/NZ02/00092

(30) Foreign Application Priority Data
May 14, 2001 (NZ) 511705

(51) Int. Cl. G01N 33/53

(52) U.S. Cl. 435/7.1

(57) ABSTRACT

A hapten-linker-large group conjugate for use in a rapid assay, wherein the assay is kinetic-based not approaching equilibrium, the hapten-linker-large group conjugate being of the general formula: X—W—Y-Z wherein: X is a hapten; W is an optional thioether or ether group;Y is a linker of 10 or more atoms in length; and Z is a large group of sufficient size to provide steric hindrance with respect to the binding of X to a ligand in the absence of Y. Also provided are processes for the production of the conjugates, assay methods and kits. The assays of the invention utilising conjugates of the invention [(5)-OVA] provide a better sensitivity than the same assays with conjugates with shorter linkers [(2)-OVA and 3-OVA].

### FIELD OF THE INVENTION

The present invention relates to small size haptens, more particularly to their conjugates with other large molecules as immobilised or mobile immunoreactants and their use in rapid assays, particularly immunoassays, especially for lateral flow tests or strip tests, flow through formats and flow immunosensors.

### BACKGROUND

Rapid lateral flow strip tests or flow immunosensors have many benefits including a user-friendly format, a relatively short processing time before receiving a test result in comparison with typical ELISA assays, and long-term stability. They are also relatively inexpensive to make.

For the immunoassay of small size haptens, such as steroids or drugs, a so-called competitive technique is usually required. As coating antigens, haptens are typically either coated onto a solid phase such as a strip membrane as a capture dot, or a line, to react with dye-, enzyme- or particle-conjugated antibodies; or labelled with dye, or other chromatically-active particles and used as mobile immunoreactants to form a colour dot or band an immunocomplex with immobilised antibodies on the solid phase. In both configurations, the coated antigens, either immobilised on the solid phase or as mobile immunoreactants, compete with free sample, antigens for a limited number of antibody binding sites. Therefore, the hapten/antibody binding capacity in the capture line is an important criteria that determines the detectable hapten concentration range and hence sensitivity of the immunoassays.

Because of their small size, it is impossible to adsorb small haptens directly onto a solid phase, such as strip membranes or particles, while retaining binding affinity to a hapten-specific antibody. Thus, small haptens have to be conjugated onto a protein for their immobilised onto a solid phase. Although there are some other materials, such as a N-vinylpyrrolidone copolymer (U.S. Pat. No. 5,723,344) or a nucleic acid fragment (U.S. Pat. No. 5,849,480) that can also be used as attachment intermediates, the conventional protein conjugations, or preparations of hapten-protein conjugates, are still the most widely used immobilisation techniques for small size haptens on solid phase (Clinica Chimica Acta, 162, 1987, 199-206).

It is generally accepted that the steric effect is one of the most important factors influencing the reactivity, or the binding specificity of antibodies with haptens. Some literature reports enhanced binding of the antibody covalently conjugated to the solid phase through an extended, flexible Streptavidin/Biotin “bridging”(Immunology Letters, 13, 1986, 313-316 and J. Immunological Methods, 111, 1988, 271-275), or through extended length linkers (Bioconjugate Chem., 7, 1996, 88-95). The antigen capture capacity of the antibody was also increased when large protein antigens were attached to solid supports through different linkers (J. Immunological Methods, 55, 1982, 1-12). The concept of hapten-spacer-protein conjugates also appears in several patents. The attachment of hapten to an immunogenic carrier protein via a spacer or linker arm may improve the immune response for the preparation of antibodies (U.S. Pat. No. 5,776,713 and U.S. Pat. No. 5,843,682). Hapten labelled with different enzymes, or other tracers, using extended linkers between the hapten and the label are more readily recognised and tightly bound by antibodies to haptens, and also maintain good enzyme activities or properties of the other tracers (U.S. Pat. No. 5,298,403; U.S. Pat. No. 5,578,457 and U.S. Pat. No. 5,834,206). Bifunctional reagents with a linker group were also used for such purpose in the synthesis of the conjugates (U.S. Pat. No. 5,595,741). It was found that construction of a drug hapten-carrier protein conjugate with a linker group have its optimal recognition by the antibodies and thus have better co-therapeutic use with other conventional drugs (U.S. Pat. No. 5,876,727). U.S. Pat. No. 5,876,727 also teaches the advantage of having a hapten displaced by a sufficient distance from a carrier or protein to allow optimal recognition of the hapten by an antibody.

Conventionally, progesterone and other similar steroid haptens with the same a ring structure have been attached to other molecules at the six and seven positions on their b ring (G. Hatzidak-is, A. Stefanakis and E. Krambovitis (1993), “Comparison of Different Antibody-Conjugate Derivates for the Development of Sensitivity and Specific Progesterone Assay” Journal of Reproduction and Fertility, 97, pages 557-561).

The applicants have now surprisingly discovered that small size hapten capture capacity of a ligand can be enhanced in a relatively short-duration assay with sensitivities for many rapid assays, particularly immunoassays, such as lateral flow strip tests or flow immunosensors, not particularly suitable for conventional ELISA by the use of a linker between the hapten and a large group to which the hapten is attached, such as a protein.

It is an object of the invention is to provide a rapid assay utilising a hapten-linker-large group conjugate, or at least to provide the public with a useful choice.

### SUMMARY OF THE INVENTION

In a first aspect, the present invention provides a hapten-linker-large group conjugate for use in a rapid assay, wherein the assay is kinetic-based not approaching equilibrium, the hapten-linker-large group conjugate being of the general formula:

X—W—Y-Z

wherein:

X is a hapten;

W is an optional thioether or ether group;

Y is a linker of 10 or more atoms in length; and

Z is a large group of sufficient size to provide steric hindrance with respect to the binding of X to a ligand in the absence of Y.

In a preferred embodiment, X is a multi-cyclic fused-ring hapten having an A-ring structure of Formula I:

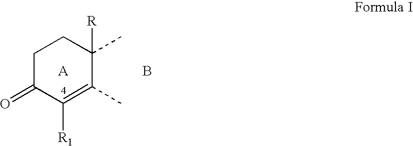

wherein R is selected from the group comprising H, CH₃ and CH₂OH and the broken lines indicate members of an adjacent B-ring structure, and R₁ is the attachment point for the linker Y.

In a more preferred embodiment, X is a hapten of Formula II:

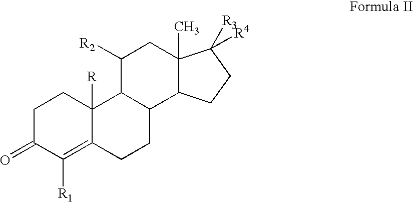

wherein:

R is selected from the group comprising: H, CH₃ and CH₂OH;

R₁ is the attachment point for the linker Y;

R₂ is H or OH;

R₃ is selected from the group comprising: oxy, C₁-C₂ alkyl, hydroxy, and methylcarbonyl, which oxy, C₁-C₂ alkyl or methylcarbonyl is optionally substituted by hydroxy; and

R₄ is hydrogen or hydroxy.

In a further aspect, the present invention provides a rapid assay method wherein the assay is kinetic-based not approaching equilibrium, the assay being for detecting a hapten in a sample, comprising the steps of:

a) contacting a ligand capable of binding the hapten with a test sample;

b) further contacting the ligand of step a) with a hapten-linker-large group conjugate of the invention specific for the ligand; and

c) determining the amount of unconjugated hapten bound to the ligand.

Preferably, the second step (b) of contacting the ligand results in contacting and binding of much of the excess unbound ligand.

Preferably, the hapten-linker-large group conjugate is immobilised.

In a preferred embodiment, the mixture of step a) is flowed over the hapten-linker-large group conjugate of step b).

In a still further aspect, the present invention provides a rapid assay wherein the assay is kinetic-based not approaching equilibrium, the assay being for detecting a hapten in a sample, comprising the steps of:

a) combining hapten-linker-large group conjugate of the invention with a test sample;

b) contacting the resultant mixture with ligand capable of binding the hapten; and

c) determining the amount of unconjugated hapten bound to the ligand.

The ligand is preferably immobilised.

In a preferred embodiment, the step b) of contacting the resultant mixture with an immobilised ligand takes place by a flow over or flow through system

In a yet further aspect, the present invention provides a rapid assay kit, wherein the assay is kinetic-based not approaching equilibrium, the kit including at least:

a ligand which binds to a hapten; and

a hapten-linker-large group conjugate of the invention.

Preferably, the kit further includes an indicator. In one embodiment, the indicator is bound to the hapten-linker-large group conjugate. In an alternative embodiment, the indicator is bound to the ligand.

Typically, kits may be flow over or flow through assay kits. A typical flow-through kit comprises a test strip.

In another aspect, the present invention provides a process for binding a hapten-linker-large group conjugate of the invention to a ligand comprising the steps of contacting the conjugate with a ligand capable of binding the hapten in the conjugate for a predetermined time where the reaction does not approach equilibrium.

The ligand is preferably immobilised. In a preferred embodiment, the ligand is preferably not bound to a hapten before being contacted by the protein-hapten conjugate.

In another aspect, the present invention provides a process for producing a hapten-linker-large group conjugate of the invention including at least the steps of:

a) mixing an activated steroid hapten dissolved in an polar organic solvent with an aqueous solution comprising 1-10 molar equivalents of a heterobifunctional water-soluble linker;

b) allow the mixture to react; and

c) attaching a large group to the remaining free functional linker group of the reaction hapten-linker product of step b).

### BRIEF DESCRIPTION OF THE FIGURES

While the present invention is broadly as defined above, it will be appreciated by those persons skilled in the art that it is not limited thereto but that it also includes embodiments of which the following description provides examples. In addition, a better understanding of the invention will be gained through reference to the accompanying drawings in which:

FIG. 1 shows the synthesis of 4-progesterone derivatives with extended length linkers.

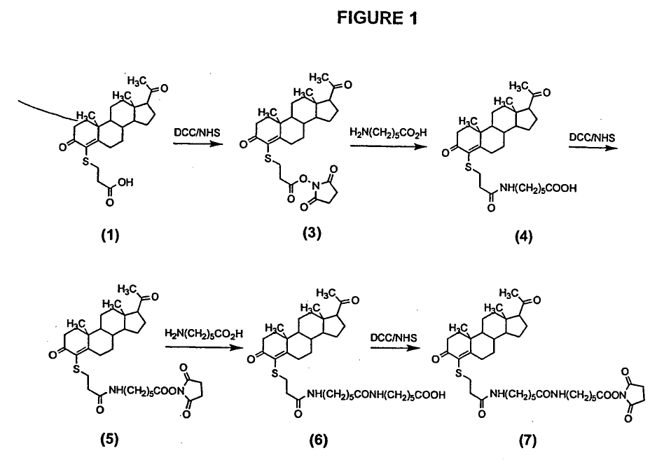

FIG. 2 shows the structures of one 7α-progesterone-OVA conjugate [(2)-OVA] and three 4-progesterone-OVA conjugates [(3)OVA, (5)-OVA and (7)OVA] with various length linkers (4-, 11-, or 18-atom linker).

FIG. 3 shows both 4- and 7α-progesterone-OVA conjugates [(2)-OVA, lower curve and (3)OVA, upper curve) having similar antibody (MoAb) binding curves.

FIG. 4 shows standard curves of antibody (MoAb) with (3)-OVA conjugate (upper curve) and (2)-OVA conjugate (lower curve).

FIG. 5 shows three 4-progesterone-OVA conjugates [(3)-OVA, lower curve; (5)-OVA, middle curve, (7)-OVA, upper curve] having similar antibody (MoAb) binding performance.

FIG. 6 shows very similar standard curves of antibody (MoAb) with the 4-progesterone-OVA conjugates [(3)-OVA, (5)-OVA, and (7)-OVA].

FIG. 7 shows different binding performance of immunogold conjugated antibody (MoAb) with three coated 4-progesterone-OVA conjugates [(3)-OVA, upper strips; (5)-OVA, middle strips; (7)-OVA, lower strips] on lateral flow test strip.

FIG. 8 shows the results of scannometric analysis [(3)-OVA, lower curve; (5)-OVA, middle curve; (7)-OVA, upper curve] of FIG. 7.

FIG. 9 shows different binding performance of three immunogold complexes with 4 -progesterone-OVA conjugates [(3)-OVA, upper strips; (5)-OVA, middle strips; (7)-OVA, lower strips] with immobilised monoclonal antibodies.

FIG. 10 shows a design of two-steps of a bio-dot flow through immunoassay format for small haptens.

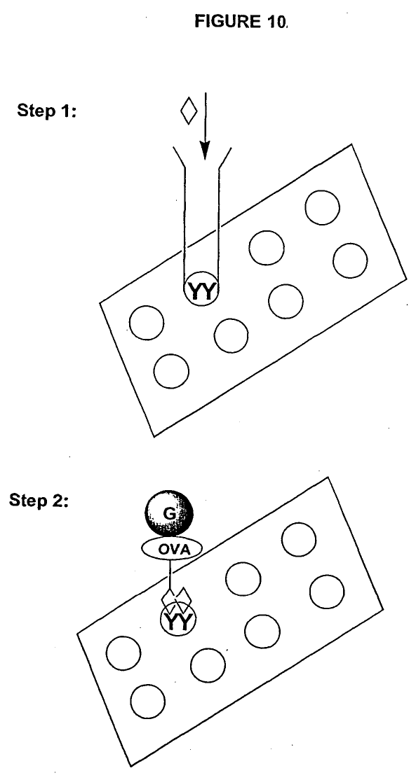

FIG. 11 shows two designs of a Surface Plasmon Resonance (SPR) flow immunosensor for small haptens using an immobilised antibody or a hapten-linker-large group conjugate of the invention onto the gold surface.

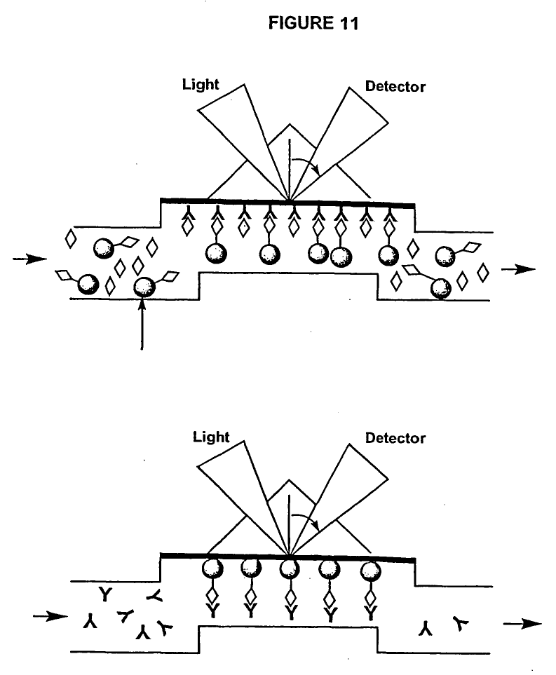

FIG. 12 shows different binding performance of four progesterone-OVA conjugates [(2)-OVA, ˜12 RU; (3)-OVA, ˜50 RU; (5)-OVA, ˜150 RU; (7)-OVA, ˜225 RU] with immobilised monoclonal anti-progesterone antibody (MoAb) by SPR biosensor.

FIG. 13 shows antibody binding responses using three progesterone-OVA conjugates [(2)-OVA, (3)-OVA and (5)-OVA] immobilised biosensor surfaces.

FIG. 14 shows standard curves of competitive immunoassay of progesterone using three progesterone-OVA conjugates [(2)-OVA, (3)-OVA and (5)OVA] biosensor surfaces.

FIG. 15 shows linear ranges of detection of progesterone obtained between 0.1 and 10 ng.ml⁻¹ from the competitive SPR-based immunoassay.

FIG. 16 depicts a process for the production of a testosterone-4-OVA conjugate with an 18-atom linker.

FIG. 17 depicts a design of synthesis of progesterone-4-OVA conjugates with various lengths of polyethylene glycol linkers.

### DETAILED DESCRIPTION OF THE INVENTION

In the present disclosure, a “rapid assay” is characterised by the assay components, when mixed, having a reaction that does not require it to approach a state of equilibrium before a meaningful result may be obtained from the assay. The invention is particularly suited to a rapid assay where the assay component reactions do not approach equilibrium during the period that the assay is run. Such rapid assays are non-equilibrium, kinetic-based assays. Typically, the assay should take less than 20 minutes to perform, more suitably less than 10 minutes.

A “large group” is a group of sufficient size to cause significant steric hindrance with respect to the binding of an attached hapten to a ligand when a linker of less than 10 atoms in length is interposed between the ligand and the hapten. Typically, in the present invention, a large group is a protein or polypeptide. Alternatively, the large group is an indicator, such as a fluorescent dye, for example bilirubin.

In one embodiment, the large group of the conjugate is a protein. The protein is any readily available protein, which is preferably inexpensive and which contains large numbers of lysine for hapten conjugations. Examples of suitable proteins in the art include bovine serum albumin (BSA), ovalbumin (OVA) or keyhole limpet hemocyanin (KLH). Proteins may include enzymes, secretory proteins, globular proteins. A preferred protein for use herein is ovalbumin (OVA). Where the protein is an enzyme, it is preferred that it be selected from the group comprising alkaline phosphatase, glucose oxidase, horseradish peroxidase and amine enriched horseradish peroxidase.

A “hapten” is a molecule selected for detection. Most usually, the hapten is a low molecular weight organic compound that reacts specifically with an antibody and which is incapable of eliciting an immune response by itself but is immunogenic when completed with an antigenic carrier. Haptens of interest here are selected from the group comprising carbohydrates, polynucleotides, steroids, steroid analogues, polypeptides (such as peptide hormones), drugs and toxins, but are not limited thereto.

Where the hapten is a steroid, such as progesterone or a molecule having an A-ring structure similar to progesterone, it is preferred that binding of the hapten occurs at the 4-position of the A-ring structure. It has been discovered that the protein conjugation site of the steroid progesterone at the C-4 position has good assay sensitivities. Previously, the literature has examined most of the common conjugation sites (3, 6β, 7α and 11α positions) of the progesterone molecule to proteins, and the most highly sensitive EIA assay results were only obtained by using either 6β or 7α positions of the steroid (Journal of Reproduction and Fertility, 97, 1993, 557-561). The 4-progesterone derivatives are simpler to synthesise and have no stereoisomeric difficulties compared with 6β or 7α analogues. This makes the 4-position of progesterone, or the 4-position of other steroids having the same A-ring structure as progesterone, an excellent alternative protein conjugation site for the 6β or 7α position. However, use of the other binding sites is not excluded.

In a preferred embodiment, the hapten is selected from the group comprising:

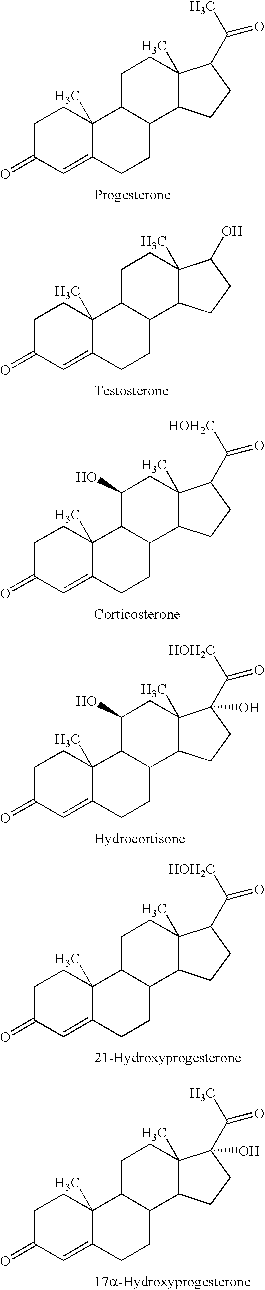

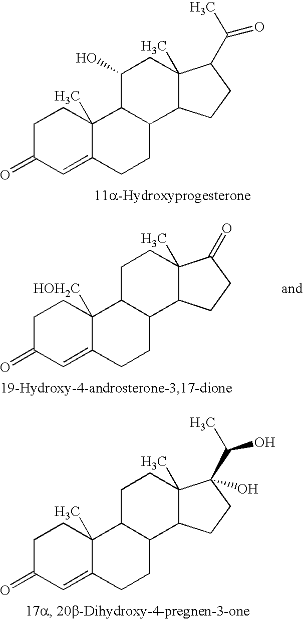

Most preferably, the hapten is progesterone.

As used herein, a “ligand” may be any molecule that has binding capacity for an antigen. It is preferably an immunoglobulin molecule capable of specific binding to a target. Such antigen targets comprise haptens as discussed above. Ligands encompass not only intact antibodies, but also fragments thereof such as Fab, (Fab)₂, Fv, single chain (ScFv), mutants thereof, fusion proteins comprising an antibody portion, and any other modified configuration of the immunoglobulin molecule that comprises an antigen recognition site of the required specificity. In an alternative embodiment, the ligand may be a T-cell receptor.

The linkers preferably comprise a chain of between 10 and 50 atoms in length, more preferably 11 to 24 atoms in length, most preferably 11 to 18 atoms in length. Any suitable linker known in the art may be employed. Linkers should be non-bulky groups proximal the hapten to ensure minimal steric hindrance wrt binding of hapten to ligand. It is also preferred that the chain be carbon-based. The carbon-based chain may also comprise one or more heteroatoms selected from N, S, O. Other side chain substituent groups may also be provided.

Preferred chains are selected from the group comprising an amino acid chain, a polyethylene glycol chain, alkyl, alkenyl, nucleic acid, and polysaccharide. Heteroatoms may be part of an amino group. The chain can have one or more sites of unsaturation, preferably 3 or less. Amino acid fragments may be incorporated into the chain. Multiple amino-acid fragments may be provided by homologation. The use of hybrid peptide-nucleic acid fragments as linkers is also contemplated.

The inventors have discovered that an optimal length of linker for reducing steric hindrance is about 18 atoms. Thereafter, minimal gains in performance are made but complications are introduced by assembly of longer linkers. Obviously, linkers longer than 18 atoms may be employed in the practice of the present invention, but they are not preferred.

In a preferred embodiment, the conjugate of the invention is represented by Formula III or by Formula IV,

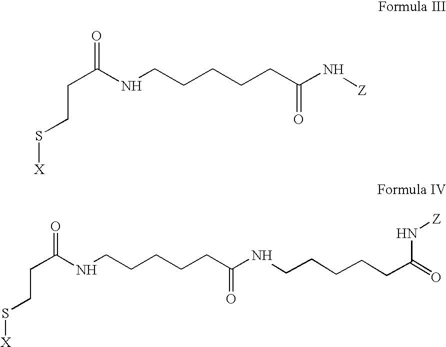

wherein X and Z are as defined above.

In one embodiment, the conjugate of the invention is:

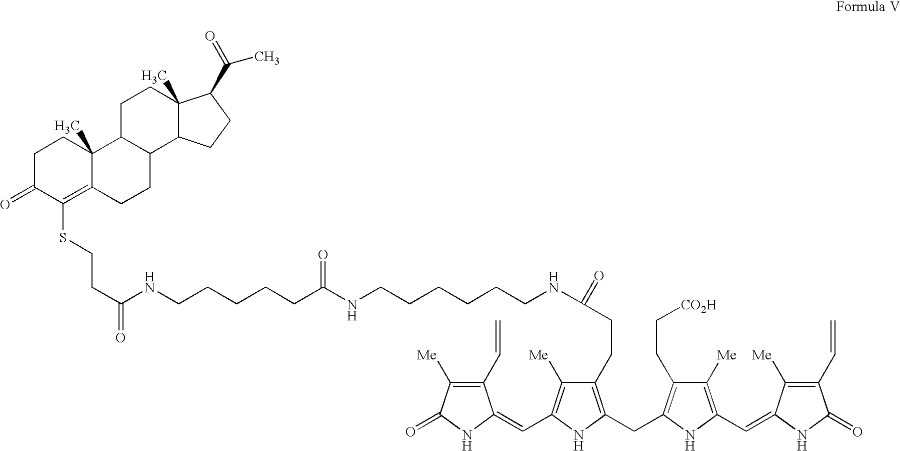

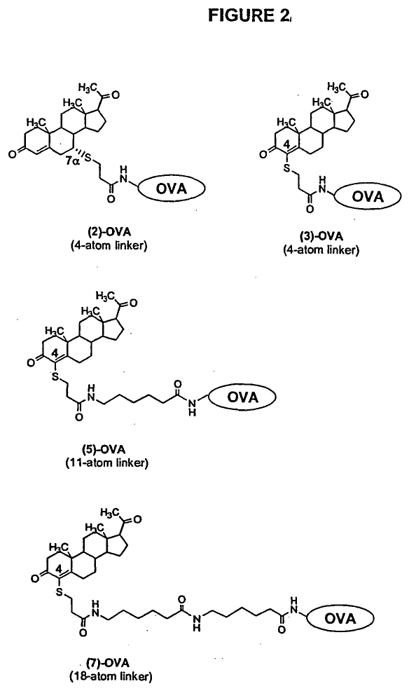

In another embodiment, preferred conjugates of the invention are (5)-OVA and (7)-OVA as depicted in FIG. 2.

The linker may be attached to the hapten and protein by any means known in the art. Preferred methods include by a covalent coupling reaction (e.g. to an amine, a carboxyl or sulfhydryl group on the protein), nucleic acid hybridisation, or ligand interaction.

In order to covalently bind steroids to linking groups, it is often necessary to include a thioether or ether bridging group, preferably a thioether group.

When a solid phase is required, the ligands or the hapten-linker-large group conjugates can be immobilised onto a solid phase. It is preferred that the amount of ligand and the hapten-linker-large group conjugate be predetermined. It is also preferred that there be an excess of protein-conjugate compared with ligands.

Both the ligand and the hapten-linker-large group conjugate can further comprise an indicator suitable for use in a detection system to enable rapid detection of the conjugate for qualitative or quantitative analysis of the hapten.

Many detection systems suitable for use in the present invention exist in the art. One having ordinary skill in the arts can detect protein binding using well-known methods. Various immunoassay procedures are described in Immunoassays for the 80's, A. Voller. et al., Eds., University Park, 1981. These include enzyme detection systems, dyes (such as fluorescent markers, and chromatic ions and complexes) and metal or non-metal colour particles (such as immunogold and coloured latex beads), and incorporated radioactive isotopes (detectable by, for example, scintillation counting).

In a preferred embodiment, immunogold particles are used because they are inexpensive and relatively stable. Suitable enzymes which may be used to detect to be label the antibody include, but are not limited to, staphylococcal nuclease, delta-5-steroid isomerase, yeast alcohol dehydrogenase, alpha-glycerophosphate dehydrogenase, triose phosphate isomerase, horseradish peroxidase, alkaline phosphatasc, asparmginase, glucose oxidase, beta-galactosidase, ribonuclease, urease, catalase, glucose-6-phosphate dehydrogenase, glucoamylase and acetylcholinesterase.

By radioactively labeling the antibody, it is possible to detect it through the use of a radioimmunoassay (RIA) (see, for example, Work, T. S, et al., Laboratory Techniques and Biochemistry in Molecular Biology, North Holland Publishing Company, N.Y., 1978). The radioactive isotope can be detected by such means as the use of a gamma counter or a scintillation counter or by autoradiography. Isotopes which are particularly useful for the purpose of the present invention are: ³H, ¹⁴C, ³⁵S, ¹²⁵I and ¹³¹I.

Fluorescent labels fall within the scope of the present invention. When a fluorescent-labelled antibody is exposed to light of the proper wavelength, its presence can then be detected due to fluorescence. Among the most commonly used fluorescent labelling compounds are fluorescein isothiocyanate, rhodamine, phycoerythrin, phycocyanin, allophycocyanin, o-phthaldehyde, fluorescamine and bilirubin.

The antibody can also be detectably labelled using fluorescence-emitting metals such as ¹⁵²Eu, or others of the lanthanide series. These metals can be attached to the specific antibody using such metal chelating groups as diethylenetriaminepentaacetic acid (DTPA) or ethylenediaminetetraacetic acid (EDTA).

The antibody also can be detectably labelled by coupling to a chemiluminescent compound. The presence of the chemiluminescently labelled antibody is then determined by detecting the presence of luminescence that arises during the course of a chemical reaction. Examples of particularly useful chemiluminescent labelling compounds are luminol, isoluminol, theromatic acridinium ester, imidazole, acridinium salt and oxalate ester.

Likewise, a bioluminescent compound may be used to label the antibody. Bioluminescence is a type of chemiluminescence found in biological systems in which a catalytic protein increases the efficiency of the chemiluminescent reaction. The presence of a bioluminescent protein is determined by detecting the presence of luminescence. Important bioluminescent compounds for purposes of labelling are luciferin, luciferase and aequorin.

Detection of an antibody may be accomplished by a scintillation counter, for example, if the detectable label is a radioactive gamma eminer, or by a fluorometer, for example, if the label is a fluorescent material.

In the case of an enzyme label, the detection can be accomplished by colorimetric methods, which employ a substrate for the enzyme. Detection may also be accomplished by visual comparison of the extent of enzymatic reaction of a substrate in comparison with similarly prepared standards.

The binding activity of an antibody may be determined according to well-known methods. Those skilled in the art will be able to determine operative and optimal assay conditions for each determination by employing routine experimentation. The methods contemplated for the present invention are kinetic-based. Equilibrium-based systems are not contemplated for the purposes of this invention.

Positive control assays may be performed in which no test compound is added.

In one embodiment, the hapten-linker-large group conjugate is as depicted in FIG. 2.

The conjugates of the invention are suitable for use in many different rapid immunoassays.

In a further aspect, the invention therefore provides a process for binding a conjugate of the invention to a ligand specific for the hapten in the conjugate. This binding step is generally conducted in the surface of the solid phase in a rapid flow over or flow through mode.

Where a solid phase is used either the ligand or the hapten-linker-large group conjugate can be immobilised. Preferably, the hapten-linker-large group conjugate is immobilised on a solid support. Solid supports which can be used within the scope of the present invention comprise primarily insoluble, polymeric materials, selected from the group consisting of polystyrene, polypropylene, polyester, polyacrylonitrile, polyvinyl chloride, polyvinylidene, polysulfone, polyacrylamide, cellulose, cellulose nitrate, cross-linked dextrans, fluorinated resins, agarose, crosslinked agarose, and polysaccharides but are not limited thereto.

In addition, however, other materials are also conceivable for use, such as glass, glass fibres, glass beads, metal, nylon mesh material, nylon membranes, metal, metal strips and metal beads. Preferred solid supports are selected from membranes, beads, microplate solid supports test tubes, microtiter plates, dipsticks, lateral flow devices, resins, PVC, latex beads and nitrocellulose. The nature of the carrier can be either soluble to some extent or insoluble for the purposes of the present invention The support configuration may be spherical, as in a bead, or cylindrical as in the inside surface of a test tube, or the external surface of a rod

Alternatively, the surface may be flat such as a sheet, test strip, etc. Those skilled in the art will know many other suitable carriers for binding antibody or antigen, or will be able to ascertain the same by use of routine experimentation.

The solid surface is more preferably a membrane.

Where the support is a metal, it is preferred that it be a metal suitable for use as a biosensor surface, such as gold or platinum.

Immobilisation on the solid support may be by passive adsorption, covalent coupling or via a ligand interaction, such as an avidin/biotin complex (for example, U.S. Pat. No. 4,467,031 Galati et al.).

The applicant has unexpectedly found that these hapten-linker-large group conjugates with longer linkers have a considerable advantage over conventional hapten-linker-large group conjugates in rapid assays. This is particularly surprising, as the performance of the conjugate is comparable with conventional conjugates when used in competitive ELISA-type assays.

Without wishing to be bound by theory, it is proposed that low sensitivities with conventional protein-hapten conjugates in rapid immunoassays may be due to steric hindrance when forming immunocomplexes with immobilised antibody. Therefore, to increase sensitivities of rapid assays, a system that minimises steric hindrance to binding rates appears to be necessary. The hapten-linker-large group conjugates of the present invention appear to achieve this end.

Without wishing to be bound by theory, the inventors proposed that the enhanced kinetics of the hapten antibody binding demonstrated in the examples of the present invention is facilitated by two key elements of the specific embodiments presented therein:

1. Binding of the linker to the 4-position of the steroid A ring; and

2. The length of the linker such that the hapten and the protein are not sterically constrained with respect to one another allowing rapid recognition and binding of the hapten by the ligand. It is believed that the hapten and the protein are afforded freedom of movement relative to one another thereby allowing rapid recognition of the binding of the hapten by the ligand.

The inventors investigated other conjugation sites besides 6β and 7α and in progesterone for immunoassays. The C-4 position of the progesterone was found by the inventors to be an excellent alternative conjugation site to the 6β-position of progesterone. Both progesterone-4-OVA (1-OVA) and progesterone-7α-OVA conjugates (2-OVA) have similar antibody-binding performance in ELISA. The progesterone-4-OVA conjugate surprisingly demonstrates better antibody binding than the progesterone-7α-OVA conjugate in kinetic-based immunoassays, such as SPR. However, compared with 6β- or 7α-progesterone derivatives, the synthesis of progesterone derivatives at C-4 position is simpler, and there is no stereoisomer problem, since the 4,5-double bond of the steroid provides only one progesterone derivative stereoisomer at the C-4 position.

Recently, it has been found that the antibody-binding signal increases with longer distances between the capture antibody and alkaline phosphatase label or the microparticle, by using extended length heterobifunctional linkers. This enhancement is regarded as a result of the increasing distance between the two entities in a general bioconjugate. The inventors have discovered that the length of linkers (4-, 11- or 18-atoms long) in the progesterone-4-OVA conjugates has no significant effect on their antibody binding performance when used as coating antigens in ELISA. The conjugates with a long linker (4- and 6-OVA) were even slightly worse than the OVA-conjugate with a short linker (1-OVA) for a short time(20 min) antibody-binding experiment. Therefore, three progesterone-4-OVA conjugates with different length linkers (4-18 atoms) have the same assay sensitivities of progesterone in ELISA.

Previous studies have shown that signal enhancement was not always proportional to the length of the linker. For example, the signal increased 64% when the length of linker was incremented from 9 atoms to 23 atoms and 82% for a 30-atom long linker in an enzyme-antibody conjugate. However, the same enzyme-antibody conjugate with a 16-atom linker had slightly better antibody-binding performance than the conjugate with a longer linker (23-atoms). These results suggest that for signal enhancement, the ideal length of the linker between the antibody and the other entity depends on the size of two entities involved and the way in which the bioconjugate is formed. In the prior art the bioconjugates were constructed between two large entities, i.e. an antibody and an enzyme or a microparticle. A linker was used only for antibody-labelling purposes, which was not directly involved in the same side of the binding-site of antibody-antigen. However, progesterone-OVA conjugates here are quite different. These were constructed with a large protein (OVA) and a very small entity i.e. a progesterone molecule (MW 314). A linker is also directly involved in the binding site of the antibody/antigen. Therefore, it is likely that the effect of length of the linker on antibody-binding performance was different from early reported studies because the bio-conjugates were formed by two different sized entities and constructed in different ways. The inventors have discovered that the length of the linker also affects antibody binding in different assay formats, particularly rapid assays.

A number of different assay types have been found to be useful in the practice of the invention. These include the test strip assays, dot-blot assays described herein (also known as the dot test or the dot immuno-binding assay), flow immunosensors such as SPR biosensor.

It is also preferred that immunoassays of the present invention take less than 20 minutes, more preferably less than 10 minutes, most preferably less than 5 minutes to complete.

In preferred test strip immunoassays, a predetermined amount of hapten-linker-large group conjugate with a long linker is immobilised onto a portion of a solid support to immobilise it. The solid support is generally cellulose-based membrane or at least a support, which allows for capillary migration of water-based solvents to occur. A test sample is applied to the membrane support and mixed with labelled antibody and rapid migrated towards the capture line, which was immobilised by hapten-inker-large group conjugate. After binding with free sample haptens the excess antibodies form a colour band with the hapten-linker-large group conjugate at the capture line. The amount of labelled antibody as ligand is quantitatively or qualitatively determined by the colour intensities of the capture line. The less the labelled antibody ligand is bound to the hapten-linker-large group conjugate of the invention, the greater the amount of hapten is in the test sample.

Other test strips may also be formulated In one embodiment, a test sample is mixed with labelled protein-hapten conjugate of the invention Both labelled hapten-linker-large group conjugate and the fire hapten migrated together towards the capture line to compete for binding sites on immobilised antibody ligands. The colour intensities, or the amount of the hapten-linker-large group conjugate, are inversely proportional to the amount of hapten in the test sample.

While any of the labels discussed above may be employed, it is preferred that the labelled conjugate be visible to the naked eye. Accordingly, a visible dye is a preferred label.

In both above strip assays, a protein conjugate of the invention has much better antibody binding in the capture line of the strip than a conjugate with a short linker. Therefore, the conjugate with a long linker can detect relatively lower antibody concentration in the capture line, thus increasing the sensitivity of the rapid strip assay.

Another embodiment of the invention provides a bio-dot flow-through immunoassay. In such an immunoassay, ligand such as antibody is immobilised on the membrane first. After sample hapten flow through the membrane through a microfiltration apparatus, the marked hapten-linker-large group conjugate of the invention react with remained antibodies to form a coloured dot on the membrane. The colour intensities of the dot, or the amount of the hapten-linker-large group conjugate, are also inversely proportional to the amount of hapten in the test sample.

Surface plasmon resonance (SPR) biosensor technology is a very useful tool for characterisation of antibody-hapten interactions. It can also be designed for a rapid immunoassay of small sized haptens. Either the hapten-linker-large group conjugate or a ligand used in SPR is usually covalently immobilised on the biosensor surface. A mixture of free sample hapten (analyte) and antibody ligand, or hapten-linker-large group conjugates, passes over the biosensor surface most usually through a microfluidic flow cell to compete for a limited number of antibody binding sites on the biosensor surface. SPR detectors monitor the change in the refractive index of the solvent layer near the surface induced by association and dissociation of the hapten-antibody complex formation. The sensitivity of SPR detectors is dependent on the refractive index of the antibody-hapten complexes on the sensor surface, which is proportional to their molecular mass. Thus, the binding of the antibody with a hapten-linker-large group conjugate gives much higher SPR response on the refractive index due to its large molecular mass of proteins than the refractive index response by antibody binding with a small size hapten.

Hapten-linker-large group conjugates of the invention demonstrate advantages in all assays tested. When the hapten-linker-large group conjugates are used as coated antigens on a solid support, immunogold conjugated antibodies can detect the hapten-linker-large group conjugate in a lower concentration for the coated conjugate with a longer linker. While for the immobilisation of antibodies, a conjugate with a longer linker also has a lower concentration detection limit of the antibody.

In strip assays, both assay configurations enhance the sensitivities of the immunochromatographic strip tests. Based on these new hapten-linker-large group conjugates with a longer linker, a highly sensitive immunochromatographic strip assay format has been developed, which can detect the small haptens at a lower concentration (<10 ng/ml) in a relatively short time. The hapten-linker-large group conjugate with a long linker of the invention is particularly useful for the strip assay when the test sample such as milk containing high content of fat, which strongly inhibits the antibody/hapten binding. Using the progesterone-OVA conjugate of the invention the lowest detection limit (LDL) of progesterone in milk is ˜10 ng/ml as judged by visual inspection, which has a lower LDL than the literature value of 30 ng/ml (Acta Chemica Scandinavica, 50, 1996, 141-145). In this literature, the strip assay has to use accurate, freshly gold-labelled progesterone-OVA conjugate for every strip assay.

For the rapid SPR (BIAcore) assay, the hapten-linker-large group conjugate with a long linker (18-atoms) gave a much better antibody binding performance (225 Response Units) than the conjugate having a short linker (4-atoms) (only 50 Response Units). The conjugate at 4-position of the progesterone molecule is also shown better antibody binding (50 Response Units) compared with the conjugate at the 7α-position of the progesterone which has the exactly same length linker (4-atoms) (only 12 Response Units).

The invention therefore also extends to test strips, kits with test strips and hapten-linker-large group conjugate reactants.

Kits of the invention comprise a solid support with immobilised ligand as described above, and a vial or container comprising labelled hapten-linker-large group conjugate of the invention.

In a still further kit embodiment, a kit comprises a solid support with immobilised hapten-linker-large group conjugate of the invention and a vial or container comprising labelled ligand to the hapten-linker-large group conjugate.

In another kit embodiment of the invention, the kit may comprise a solid support with immobilised ligand as described above and a vial of container comprising a conversion kit for converting a hapten to a hapten-linker-large group conjugate of the present invention.

Most usually, the kit also comprises a solvent, preferably an aqueous solvent, for migrating a test sample and the labelled conjugate over the solid support. Where the labelled conjugate is not visible to the naked eye, means may be provided in the kit for detecting the presence of the labelled conjugate. The kits would also usually comprise applicators, preferably for delivering a predetermined volume of sample and/or conjugate and/or ligand to the solid support.

The hapten-linker-large group conjugates of the present invention may be produced by:

1. binding a hapten to a linker as defined above. Preferably, the binding is through chemically covalent coupling reaction; or it may be by nucleic acid hybridisation where the linker is a nucleic acid.

2. binding the free end of the linker to a large group, such as a protein. Known processes for binding linkers to proteins may be selected from the standard reference (Bioconjugation: Protein Coupling Techniques for the Biomedical Sciences, Edited by Mohammed Aslam and Alartair Dent)

Alternatively, it is possible to first bind the linker to the protein and then to the hapten by the same processes.

A process for producing a hapten-linker-large group conjugate of the invention is provided, including at least the steps of:

d) mixing an activated steroid hapten dissolved in an polar organic solvent with an aqueous solution comprising 1-10 molar equivalents of a heterobifunctional water-soluble linker,

e) allow the mixture to react; and

f) attach a large group to the remaining free functional linker group of the reaction hapten-linker product of step b).

The process may optionally include an isolation step between steps b) and c) for isolating the hapten-linker product.

Preferably, the final mixture has an aqueous content of between 2 and 30%, more preferably between 5 and 15%, most preferably 10%.

Step b) reaction time is preferably in the order of up to 24 hours, more preferably, less than 12 hours, most preferably 3 hours. The reaction in b) generally takes place at room temperature. In a preferred embodiment, the reaction in b) takes place at substantially neutral pH.

Preferably, the aqueous solution of step a) comprises 2-5 molar equivalents of a heterobifunctional water-soluble linker.

In one embodiment, the activated steroid is an active ester of the steroid. In a most preferred embodiment, it is a succinimide ester of the steroid.

Preferably, the heterobifunctional linker has two different functional groups selected from the group comprising: carboxyl, amino, thiol, hydroxy, aldehyde and reactive halide. In a preferred embodiment, the heterobifunctional groups are carboxyl and amino.

Any non-reactive polar organic solvent may be used in the reaction. The polar organic solvent may be conveniently selected from DMF, DMSO, acetone and THF.

Industrial Application

Thus in according with the present invention, there are provided assays for detecting haptens in a test sample. The conjugates are particular useful where the haptens are in low concentrations and/or containing high content fat in test samples that conventional immunoassays do not sufficiently or can not rapidly detect them. The assays are particularly useful when formulated for in situ testing. One such commercial application is for rapid, on-site detection of milk progesterone at low levels in cattle, which can be used for pregnancy diagnosis and oestrus detection of the cattle.

The invention will now be illustrated with reference to the following non-limiting examples:

### EXAMPLE 1

### Synthesis of Four progesterone-OVA Conjugates with Barious Length Linkers

(a). Progesterone 4-mercaptopropionic acid (1)- and 7α-mercaptopropionic acid (not shown). These Compounds were prepared according to a literature procedure (Steroids, 53, 1989, 727-738) for the preparation of 11α-hydroxyprogesterone 4- and 7α-mercaptopropionic acids.

(b). Succinimidyl progesterone 4-mercaptopropionatey (3). To a 3 ml dry DMF solution of progesterone 4-mecaptopropionic acid 1 (434.8 mg, 1.04 mmol) a solution of dicyclohexyldiimide (235.7 mg, 1.14 mmol) in 1.0 ml of DMF was added, followed by N-hydroxysuccinimide (131.5 mg, 1.14 mmol) in 1.0 ml of DMF. The solution was stirred at room temperature overnight. Pure product (3) was obtained after flash column chromatography.

(c). Synthesis of succinimidyl progesterone 4-mercaptopropionates (5). Progesterone 4-succinimidyl ester 3 (300 mg, 0.58 mmol) was dissolved in 4 ml of dry DMF. To this solution was added ε-6-aminocaproic acid (229 mg, 0.91 mmol) in 0.3 ml of deionised water, and the reaction mixture was stirred at room temperature overnight. After purification, the reaction gave a white solid product 4 (63.6% yield) as a single spot on a TLC plate, which was used for the preparation of its succinimidyl ester 5 directly without any further purification. Compound 5 was prepared in a manner analogous to the preparation of 3

(d). Synthesis of succinimidyl progesterolie 4-mercaptopropionates (7). Compound 6 was prepared in a similar manner to the preparation of 4 from 3. With compound 5 (157.6 mg, 0.25 mmol) and ε-6-aminocaproic acid (98.7 mg, 0.39 mmol) as starting material, compound 6 (57% yield) was obtained after column flash chromatography. Compound 7 was also prepared in a manner analogous to the preparation of 5. With compound 6 (92.1 mg, 0.14 mmol) as starting material, product 7 was obtained as a clear, colorless oil after column flash chromatography. All the above synthesized progesterone derivatives (1˜7) were confirmed by ES-MS and NMR analysis.

(e). Protein conjugations. A series of progesterone-ovalbumin conjugates were prepared from either progesterone free acids (including 1), or the isolated progesterone succinimidyl esters (3, 5 and 7) as follows:

To a solution of compound 1 (29.5 mg, 0.07 mmol) in DMF (0.5 ml) were added dicyclohexyldiimide (16.02 mg, 0.078 mmol) in 0.1 ml of DMF and N-hydroxysuccinimide (8.94 mg, 0.078 mmol) in 0.1 ml of DMF. The solution was stirred at room temperature for 90 min. after which time the solution was added to a solution of OVA (63.5 mg, 0.0014 mmol) in phosphatc buffer (7 ml, pH, 7.0). The conjugation reaction was left stirring at 4° C. overnight. The samples were then dialyzed against aqueous NaHCO₃, deionised water, and PBS/T. After further purification with a PD-10 column, the amount of protein in these purified samples, as determined by the BCA assay, was found to be 4.11 mg ml⁻¹ for progesterone-4-OVA, and 4.02 mg ml⁻¹ for progesterone-7α-OVA, respectively.

The isolated, pure progesterone succinimidyl esters (3, 5, and 7) were also used for the protein conjugations directly. A solution of 0.019 mmol of progesterone active ester (3, 5 or 7) in 0.2 ml of DMF or DMSO was slowly added to a solution of ovalbumin (17.1 mg, 0.38×10⁻³ mmol) in phosphate buffer (15 ml, pH, 7.0) while stirring. The purification of these three protein conjugates was performed in the same procedure as previously described above. Th amount of protein in these progesterone-4-OVA conjugates, as determined also by the BCA protein assay, was found to be 1.09 mg ml⁻¹ (4 atoms linker), 3.56 mg ml⁻¹ (11 atoms linker), and 4.12 mg ml⁻¹ (18 atoms linker) respectively. The average conjugation degrees for the progesterone-OVA conjugates, or the hapten numbers per protein, are estimated to be four on average for each conjugate, which were directly determined by MALDI TOF mass spectrometry. The structures of these progesterone-OVA conjugates are shown in FIG. 2.

### EXAMPLE 2

### Direct or Competitive ELISA Using progesterone-OVA Conjugates as Coating Antigens

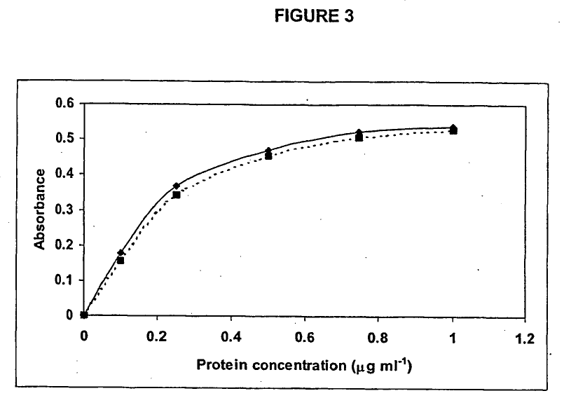

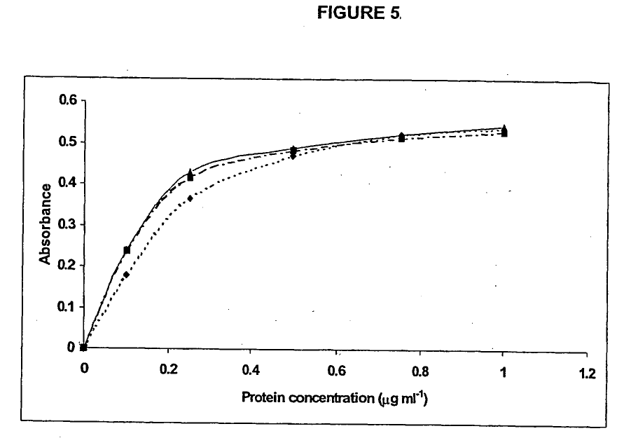

A direct ELISA determined the binding reactivity of progesterone-OVA conjugates to MAb. The microtitre plates were pre-coated with progesterone-4- or -7α-OVA conjugates in a series of concentrations in NaHCO₃ (50 mM, pH 9.6). MAb solution in PBS/T (5 μg ml⁻¹, 100 μl per well) was added. The plates were shaken for 3 hours at room temperature and left at 4° C. overnight. The plates were then added peroxidase-labeled anti-rat IgG solution. Finally, 200 μl of substrate solution were added into each cell, and the enzymatic reaction was stopped after 30 min by addition of 50 μl of 1 M H₂SO₄. The plates were then read and recorded at 450 nm after 15 minutes. The antibody binding curves for both 4-and 7α-progesterone-OVA conjugates are shown in FIGS. 3 and 5.

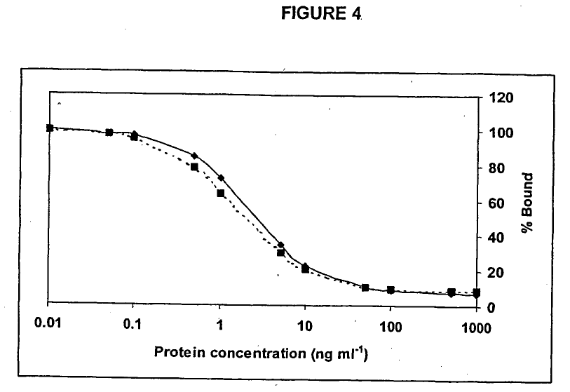

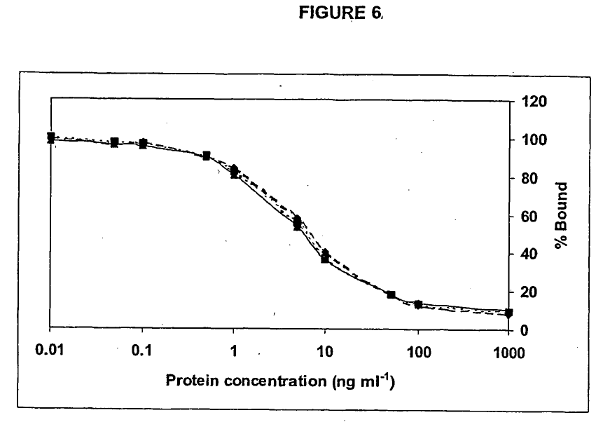

Standard curves (see FIGS. 4 and 6) were produced using progesterone-OVA conjugates in NaHCO₃ (50 mM, pH 9.6) as coating antigens (1 μg ml⁻¹, 100 μl per well). Various standard progesterone solutions (0.001˜1000 ng ml⁻¹) in PBS/T (100 μl per well) and MAb solution (1 μg ml⁻¹) in PBS/T (100 μl per well) were added to the plates at the same time for competitive ELISA. All the other ELISA procedure was performed in the same fashion as that for the direct ELISA.

It has been found by the inventors that the progesterone-OVA conjugates with different length linkers and different conjugating positions (4- or 7α) on progesterone have no significant effect on the antibody binding performance by conventional ELISA.

### EXAMPLE 3

### Preparation of Immunogold Complexes with Monoclonal Antibody (MoAb), or with 4-progesterone-OVA Conjugates

(a). Preparation of colloidal gold particles (G40). The preparation was performed according to a similar literature (Analyst, 123, 1998, 2437-2441).

(b). Preparation of monoclonal antibody-, or 4-progesterone-OVA conjugates-coated colloidal gold. After titration of the colloidal gold (G40) solution to pH=7.7 with K₂CO₃ (0.02 M), antibody (120 μg) or the 4-progesterone-OVA conjugates (60 μg) solution (0.2 ml) was added into the pH adjusted colloidal gold solution (10 ml) with vigorous vortex mixing. The immunogold solutions were then blocked with 20% OVA solution in PBS (0.15 ml). After repeated washing with 2% OVA and centrifugation at 6.000 rpm, the inmnunogold solutions were stored in borate (NaB₇O₄) buffer (2 mM, pH=7.2) containing 0.1% NaN₃ at 4° C.

### EXAMPLE 4

### Immunochromatographic Strip Tests Using 4-progesterone-OVA Conjugates as Coating Antigens, or Immobilising Monoclonal Antibody onto the Test Strip

Nitro-cellulose membrane (AE 100, Schleicher & Scbuell, Germany) laminated with polyester backing support (GL-187, G&L Precision Die Cutting, Inc. USA) was cut into strips (4 or 5 mm wide). After blocked with 0.01% Tween-20 solution in water for 10 min, and followed by drying at 40° C. for 2 hours, the strips were coated with either anti-progesterone MAb solution (0.5 μl per strip), or 4-progesterone-OVA conjugate solutions (0.5 μl per strip), the strips were again dried at 40° C. for 2 hours. The strips were stored in a silica get container overnight.

(a). For strip coating with different concentrations of OVA-progesterone conjugate solutions, the running solvents for half strip in a microwell are as follows:

15 μl of 1% Tween-20 in PBS

15 μl of 2% polyvinyl pyrolidine (PVP, MW 44000) in PBS

3 μl of antibody-immunogold solution (0.72 μg MAb per strip).

(b). For strip coating with the different concentrations of monoclonal antibody solutions, the running solvents in a microwell are as follows:

20 μl of 2.5% Tween-20 in PBS

20 μl of 2% polyvinyl pyrolidine (PVP, MW 44000) in PBS

5 μl of progesterone-OVA coated immunogold (0.3 μg protein per strip).

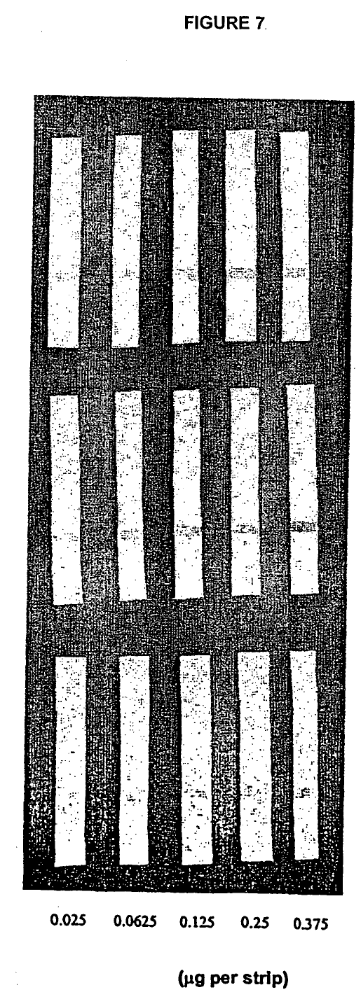

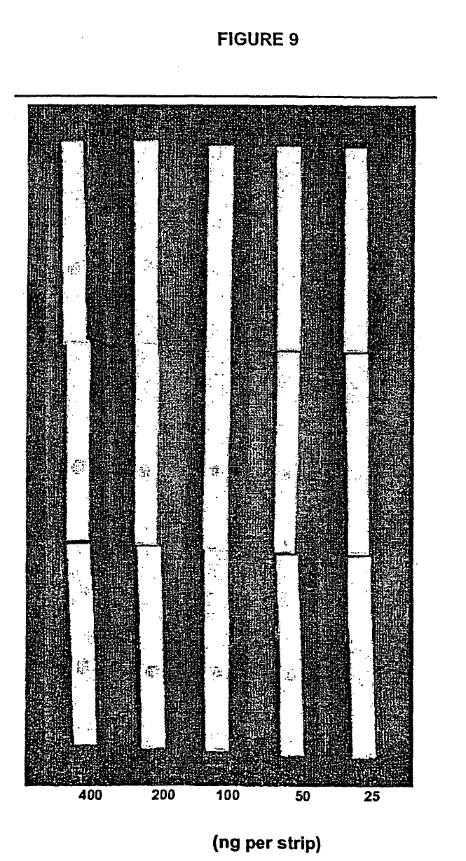

The antibody binding performance in above two different configurations are shown in FIG. 7 and FIG. 9.

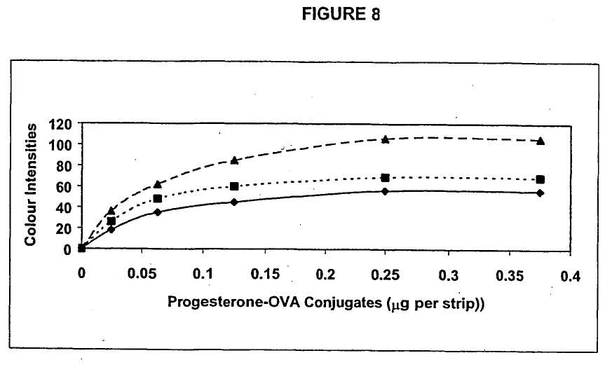

(c). Scanning Analysis. After running the strips the colour intensities of different test strips in FIG. 8 were analysed by scanning analysis using “Image Tool, Version 2.00 ”, which is free from the intent, according to following procedure:

1. Placed strips onto scanner bed

2. Opened Image Tool and selected file, acquire, TWAIN

3.

| | | |
|---|---|---|
| Settings: | Scaling = | 36% |
| | Width = | 3.31 cm |
| | Height = | 3.49 cm |
| | Resolution = | 400 |

4. Pressed preview and once finished pressed Scan

5. Saved colour image as JPG file and maximum resolution

6. Converts image into 8 bpp black and white image by selecting “Processing” and “Colour to Greyscale”

7. Selected “Analysis”and “Line Profile”, dragged line over band trying to get the ends of the line on the white surface (repeat three times for one band and take the average value).

8. “Image Tool” automatically recorded the entire peak height values which wore related the colour intensities on the strips.

### EXAMPLE 5

### Evaluation of Antibody Binding Performance Using Monoclonal Antibody Biosensor Surface

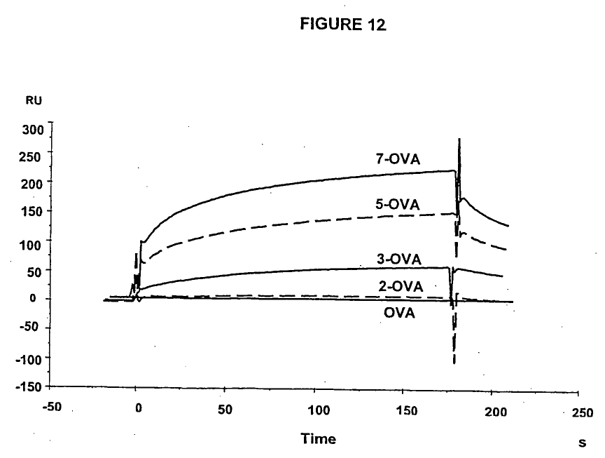

A biosensor surface immobilising by antibody solution (100 μg/ml in HBS buffer) was prepared by standard amine coupling of antibody to an activated CM-5 sensor chip. Four different progesterone-OVA conjugates [(2)-OVA, (3)-OVA, (5)-OVA, and (7)-OVA] (at 1 mg/ml concentration in HBS buffer) were injected serially over the calibrated biosensor surface at 10 μl/min for 3 minutes. The binding responses (Response Units or RU) were measured for each conjugate, which is shown in FIG. 12. The results of binding affinities for the conjugates have clearly indicated that the conjugate at 4-position of the progesterone is better than the 7α-progesterone derivative, and also the conjugate having a long linker gave a far better antibody binding than the conjugate with a short linker.

### EXAMPLE 6

### Evaluation of Antibody Binding Performance Using progesterone-OVA Conjugate Biosensor Surfaces

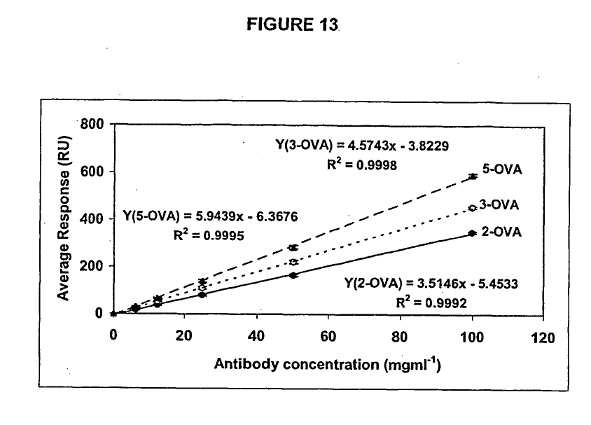

Antibody binding studies were also evaluated using the above three progesterone-OVA conjugates [(2)-OVA, (3)-OVA and (5)-OVA] biosensor surfaces. Specifically, six concentrations of MoAB (0-100 μg.ml⁻¹) were individually injected over the blank and immobilized progesterone-OVA conjugate surfaces. Data from each conjugate surface was also corrected by subtraction of the sensorgram data from the blank surface. Three identical calibration studies were performed with no significant changes in the binding response for each of the antibody concentrations. Therefore, three calibration curves for 1-, 2- and 4-OVA were obtained by taking average binding responses for each concentration of the MoAB, and plotted against the antibody concentrations (FIG. 13). The antibody-binding signal of the progesterone-4-OVA conjugates was increased by 30% as the length of the linker was increased from a 4-atom to an 11-atom linker. The signal enhancement was also improved by 31% for progesterone-4-OVA conjugate [(3)OVA] compared with the 7α-conjugate [(2-OVA].

### EXAMPLE 7

### Competitive progesterone Immunoassay by SPR Using progesterone-OVA Conjugates Biosensor Surfaces

For development of a competitive SPR immunoassay for progesterone, a progesterone-OVA conjugate is immobilized on the biosensor surface to give better binding responses, compared to an antibody-immobilized biosensor surface. From screening tests using different progesterone-OVA conjugates on the antibody biosensor surface (FIG. 12), the conjugate [(7)-OVA] with a linker of 18-atoms had the highest antibody-binding signal (225 RU). This appears best as a sensor probe on the surface for design of a competitive BIAcore assay. However, this conjugate [(7)-OVA] failed to immobilize on the sensor surface, and we can only immobilize the other progesterone-OVA conjugates [(2)-OVA, (3)-OVA and (5)-OVA] on the sensor surfaces for competitive BIAcore assays.

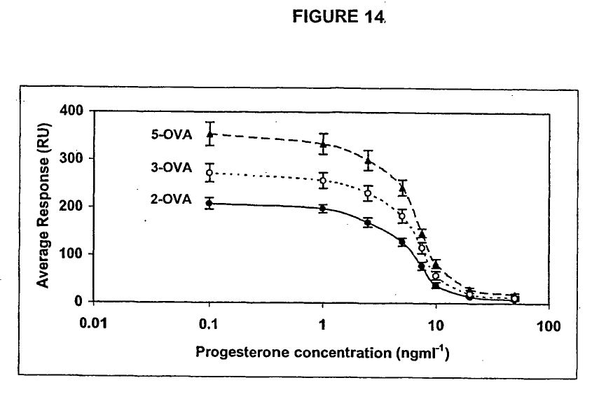

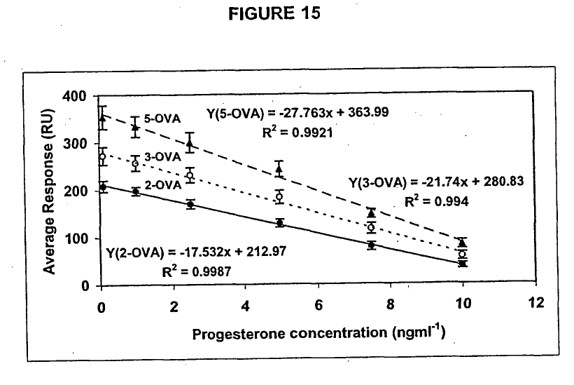

After mixing a fixed concentration of MoAB (200 μg.ml⁻¹) with each of a series of concentrations of free progesterone in HBS buffer, the remaining antibody in each equilibrium mixture was subsequently injected on to the progesterone-OVA conjugate biosensor surfaces and antibody binding responses measured. The same experiments were repeated five times with good reproducibility for each concentration of progesterone. The binding of antibody to the surface of the chip was inversely proportional to the amount of free progesterone in solution. A plot of concentrations of free progesterone versus average response (RU) of antibody binding provided three standard competition curves for three progesterone-OVA conjugates (FIG. 14). The assays demonstrated good sensitivities over the range 0-50 ng.ml⁻¹ of progesterone concentrations. The immunoassay sensitivity is normally determined by the steepness of the calibration curve, i.e. response per unit concentration. Therefore, the [(5)-OVA] conjugate has shown a better assay sensitivity than the [(3)-OVA] conjugate, which is slightly more sensitive than the [(2)-OVA] conjugate. The results have clearly indicated the effects of the linker of the conjugates and the conjugation positions in the steroid molecule for SPR-based immunoassay. The SPR-based assays also exhibited a linear range of detection between 0.1 and 10 ng.ml⁻¹, and the R² values for this range were found to be over 0.99 for all three progesterone-OVA conjugates (FIG. 15). The SPR-based immunoassays were fully competitive with conventional ELISA techniques but much more rapid and simple.

### EXAMPLE 8

### Determination of Milk progesterone Concentration by Immunochromatographic Strip Tests Using 4-progesterone-OVA Conjugate with a Long Linker (18-Atoms)

The 4-progesterone-OVA conjugate with a long linker (18-atoms) of the invention can be coated onto different membrane strips. The previously prepared antibody-gold conjugate can be diluted with 1-5% sucrose solution in water. The different conjugate pads can be cut into small size, and the above diluted immunogold solution (˜10 μl) can be applied to each conjugate pad by soaking the gold solution into the sheet. The gold conjugate pad will be dried, and stored.

A lower wick material, or glass fibre, as a sample pad is saturated with Tris-buffer with 1-5% Tween-20 in water overnight and dried before use. All the test ingredients (membrane, gold conjugate pad, sample pad and solvent absorbent pad) can be pasted onto the backing plate and mounted in the plastic housing. This ready to use strip test device will be relatively stable when it is sealed in a pouch in the presence of a bag of drying agent.

This rapid lateral flow strip test can be successfully applied to measuring farm milk progesterone (P₄). The strip is a convenient alternative to conventional ELISA. It is also much faster than ELISA-based tests (10 minutes or less). It is particularly suited for on-site use by farmers. The test can be used for cow pregnancy diagnosis (For progesterone <10 ng/ml, an intensive colour band will show a negative pregnancy result; while for progesterone >10 ng/ml, a weak colour band or no colour band will show a positive pregnancy result).

### EXAMPLE 9

### Synthesis of Testosterone-4-OVA Conjugate with an Extended Linker

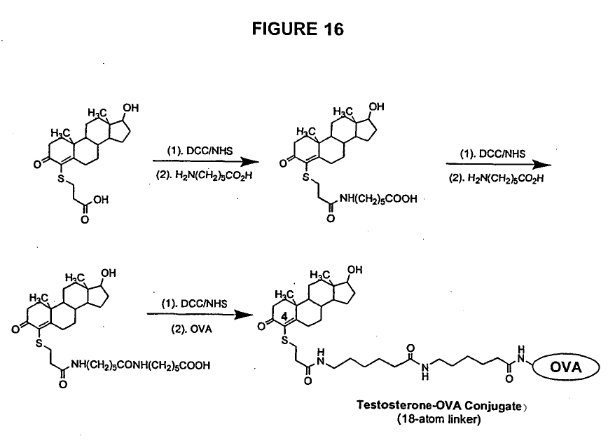

The synthesis of testosterone-4-OVA conjugate with an 18-atom linker and its OVA conjugation will be carried out in the same procedure as shown in Example 1 using testosterone as steroid hapten instead of using progesterone. The process for the production of the testosterone-4-OVA conjugate is depicted in FIG. 16.

The above procedure can be applied to other steroids having the same A-ring structure to progesterone molecule, such as testosterone, corticosterone, 17α,20β-dihydroxy-4-pregnen-3-one, 11α-hydroxyprogesterone, 17α-hydroxyprogesterone, 21-hydroxyprogesterone, 19-hydroxy-4-androstene-3,17-dione and hydrocortisone.

### EXAMPLE 10

### Synthesis of progesterone-OVA Conjugates with Various Length of polyethylene glycol (PEG) Linkers

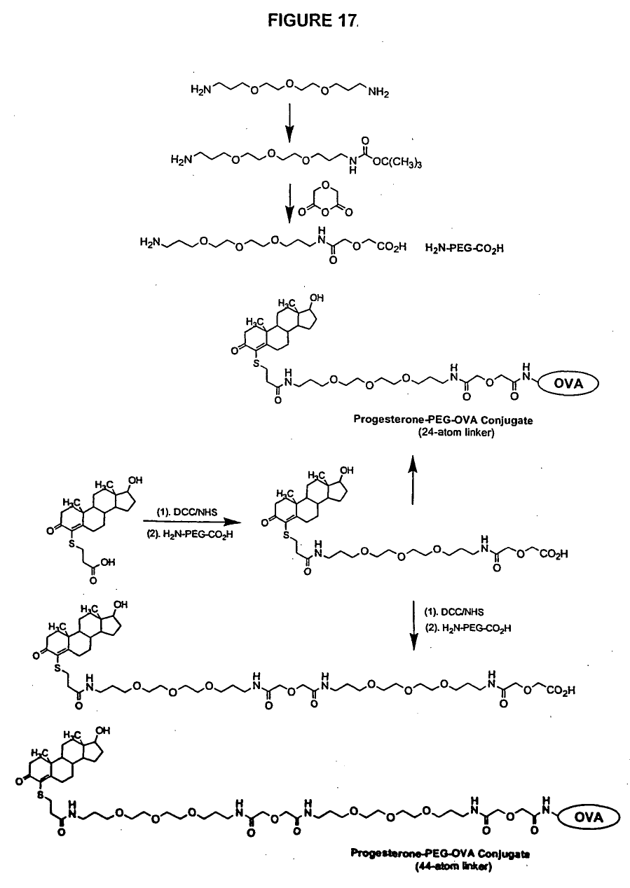

The progesterone-OVA conjugates can be also synthesised using different types of linkers such as polyethylene glycol (PEG) (FIG. 17) as shown by following steps:

(a). Heterobifuiictioiial PEG linker (H₂N—PEG-CO₂H). Mono-protected PEG amine compound can be prepared by reaction of a commercial PEG diamine with di-tert-butyl dicarbonate. The resulted mono-protected product can be reacted with a commercial dicarboxylic acid anhydride, followed by de-protection of amine to provide a heterobifunctional PEG amino acid linker with 24-atoms in length, which has the carboxylic acid group at one end, and the amine group at the other end of the linker.

(b). Synthesis of progesterone-PEG-OVA conjugates with various length linkers. The progesterone-4-mercaptopropionic acid (1) can be converted into the active succinimidyl ester as the same procedure as in Example 1. The resulted activated steroid (3) can be reacted with the above PEG amino acid linker (H₂N—PEG-CO₂II) to form progesterone PEG derivative with a longer linker (44-atoms in length). The progesterone PEG derivatives (24- and 44-atom linkers) can be coupled to the OVA proteins via the similar protein conjugations as shown in the Example 1.

The Progesterone-PEG-OVA conjugates are especially suitable for construction of protein conjugates with long linkers, which have much better water solubility than the conjugates with simple aminocaproic acid chain [(3)-OVA, (5)-OVA and (7)-OVA].

### EXAMPLE 11

### A Rapid Immunoassay by Size Exclusion Chromatography

This example describes a process for performing a rapid immunoassay by size exclusion gel column chromatography. There is no solid phase immobilization. Instead of using a protein, as in the examples above, progesterone is conjugated with a fluorescent dye via an extended linker to form the progesterone-linker-bilirubin of Formula V.

The rapid immunoassay should be carried out as follows:

Step 1: A fixed amount of the above progesterone-linker-bilirubin conjugate of Formula V and a predetermined amount of anti-progesterone antibody should be well mixed with a sample solution whose progesterone concentration is to be determined. The mixture should be permitted to form immunocomplexes.

Step 2: The above mixtures quickly flow through a short size exclusion column, which will be monitored by HPLC with an UV detector at ˜450 nm. Two major peaks will be detected in the HPLC chromatograph. The first peak will represent immunocomplex of the antibody with progesterone-linker-bilirubin conjugate, which will elute early, due to its much higher mass (>150,000), while the second peak represented the progesterone-linker-bilirubin conjugate has much lower mass (<1,000) and will elute later. No other components show up on the chromatogram since there is no fluorescent dye attached to any other species.

The first peak will be representative of the amount of progesterone-bilirubin conjugate and antibody immunocomplex. The second peak will be representative of the amount of progesterone-bilirubin conjugate not bound in an immunocomplex.

If the concentration of free progesterone in a given sample is high, then the limited antibodies form less immunocomplex with the progesterone-bilirubin conjugate. This causes the first peak to be smaller than the second peak. In contradistinction, if the concentration of free progesterone in a given sample is low, then the limited antibodies form more immunocomplex with the progesterone-bilirubin conjugate. This causes the first peak to be larger than the second peak. Therefore, the area of the first peak, or the ratio of the first peak area to the second peak will be inversely proportional to the sample progesterone concentrations.

It will be appreciated by art skilled workers that the above disclosure is provided by way of example only. Many different variations of the present invention may be made.

1. A hapten-linker-large group conjugate for use in a rapid assay, wherein the assay is kinetic-based not approaching equilibrium, the hapten-linker-large group conjugate being of the general formula:
X—W—Y-Z
Wherein:
X is a hapten;
W is an optional thioether or ether group;
Y is a linker of 10 or more atoms in length; and
Z is a large group of sufficient size to provide steric hindrance with respect to the binding of X to a ligand in the absence of Y.

2. A hapten-linker-large group conjugate of claim 1, wherein W is a thioether bridge.

3. A hapten-linker-large group conjugate of any one the preceding claims, wherein Y is of between 10 to 50 atoms inclusive in length.

4. A hapten-linker-large group conjugate of any one of the preceding claims, wherein Y is of between 11 to 24 atoms inclusive in length.

5. A hapten-linker-large group conjugate of any one of the preceding claims, wherein Y is of between 11 to 18 atoms inclusive in length.

6. A hapten-linker-large group conjugate of any one of the preceding claims, which is: 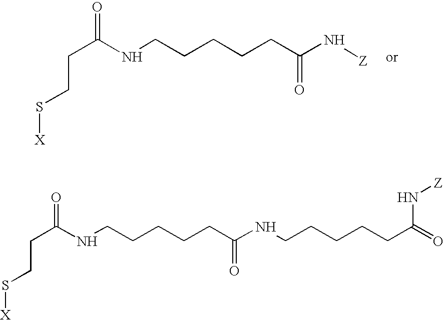
wherein X and Z are as defined in claim 1.

7. A hapten-linker-large group conjugate of any one of the preceding claims wherein Z is a protein or a polypeptide.

8. A hapten-linker-large group conjugate of any one of the preceding claims wherein Z is ovalbumin.

9. A hapten-linker-large group conjugate of any one of 1 to 0, wherein Z is an indicator group.

10. A hapten-linker-large group conjugate of any one of 1 to 0 and 9, wherein Z is bilirubin.

11. A hapten-linker-large group conjugate of any one of the preceding claims, wherein X is a steroid or steroid analogue.

12. A hapten-linker-large group conjugate of any one of the preceding claims wherein X is a multi-cyclic fused-ring hapten having an A-ring structure of Formula VI: 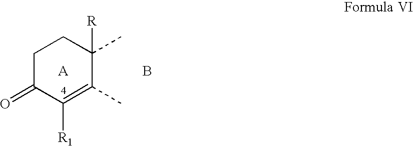
wherein R is selected from the group comprising H, CH₃ and CH₂OH and the broken lines indicate members of an adjacent B-ring structure and R₁ is the attachment point for the linker Y.

13. A hapten-linker-large group conjugate of any one of the preceding claims, wherein X is a hapten of Formula VII: 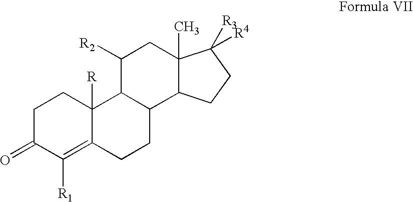
wherein:
R is selected from the group comprising: H, CH₃ and CH₂OH;
R₁ is the attachment point for the linker Y;
R₂ is H or OH;
R₃ is selected from the group comprising: oxy, C₁-C₂ alkyl, hydroxy, and methylcarbonyl, which oxy, C₁-C₂ alkyl or methylcarbonyl is optionally substituted by hydroxy, and
R₄ is hydrogen or hydroxy.

14. A hapten-linker-large group conjugate of any one of the preceding claims, wherein X is selected from the group comprising:

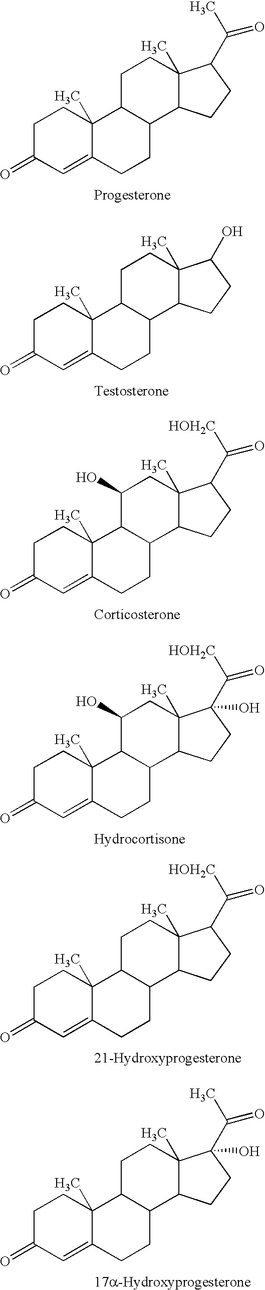

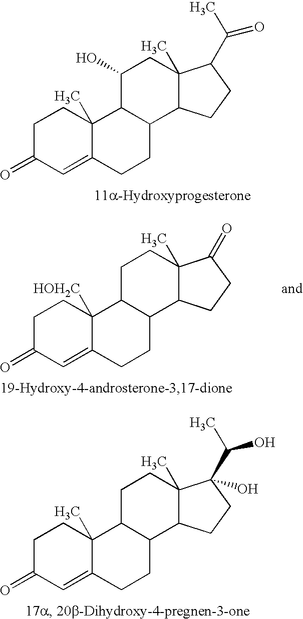

15. A hapten-linker-large group conjugate of any one of the preceding claims, wherein X is progesterone.

16. A hapten-linker-large group conjugate of claim 10 that is:

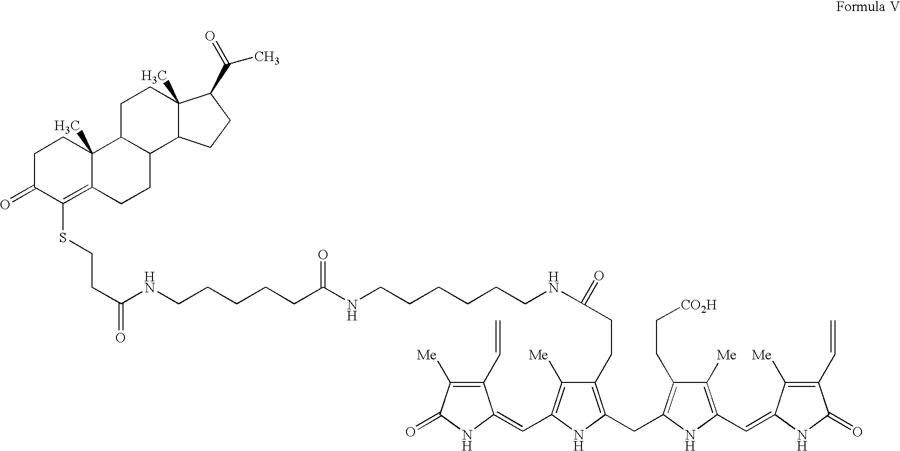

17. A hapten-linker-large group conjugate of any one of claims 1 to 9 and 11 to 15 that is: 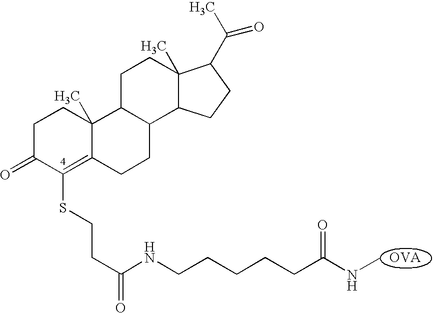
wherein OVA is ovalbumin.

18. A hapten-linker-large group conjugate of any-one of claims 1 to 9 and 11 to 15 that is: 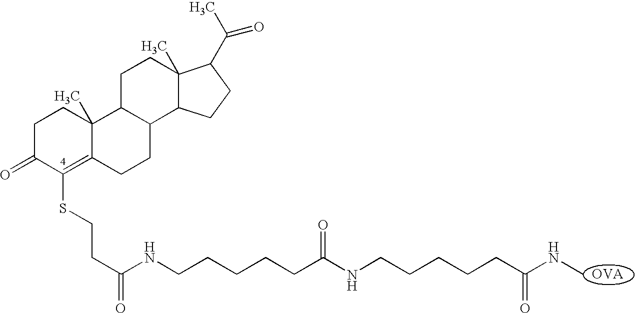
wherein OVA is ovalbumin.

19. A hapten-linker-large group conjugate of any one of the preceding claims, wherein the ligand is an immunoglobulin molecule.

20. A hapten-linker-large group conjugate of claim 18, wherein the ligand is an antibody or an antibody fragment.

21. A rapid assay method wherein the assay is kinetic-based not approaching equilibrium, the assay being for detecting a hapten in a sample, comprising the steps of:
d) contacting a ligand capable of binding the hapten with a test sample;
e) further contacting the ligand of step a) with a hapten-linker-large group conjugate of any one of claims 1 to 20 specific for the ligand; and
f) determining the amount of unconjugated hapten bound to the ligand.

22. A rapid assay method according to claim 21, wherein the second step (b) of contacting the ligand results in contacting and binding of much of the excess unbound ligand.

23. A rapid assay method according to claim 21 or claim 22, wherein the hapten-linker-large group conjugate is immobilised.

24. A rapid assay method according to any one of claims 21 to 23, wherein the mixture of step a) is flowed over the hapten-linker-large group conjugate of step b).

25. A rapid assay method according to any one of claims 21 to 24, wherein the hapten is a steroid.

26. A rapid assay method according to any one of claims 21 to 24, wherein the hapten is progesterone.

27. A rapid assay method according to any one of claims 21 to 25, wherein the ligand is an antibody.

28. A rapid assay wherein the assay is kinetic-based not approaching equilibrium, the assay being for detecting a hapten in a sample, comprising the steps of:
a) combining hapten-linker-large group conjugate of any one of claims 1 to 20 with a test sample;
b) contacting the resultant mixture with ligand capable of binding the hapten; and
c) determining the amount of unconjugated hapten bound to the ligand.

29. A rapid assay according to claim 28, wherein the ligand is immobilised.

30. A rapid assay according to claim 28 or claim 29, wherein the step b) of contacting the resultant mixture with an immobilised ligand takes place by a flow over or flow through system.

31. A rapid assay method according to any one of claims 28 to 30, wherein the hapten is a steroid.

32. A rapid assay method according to any one of claims 28 to 31, wherein the hapten is progesterone.

33. A rapid assay method according to any one of claims 28 to 32, wherein the ligand is an antibody.

34. A rapid assay kit, wherein the assay is kinetic-based not approaching equilibrium, the kit including at least:
a) a ligand which binds to a hapten; and
b) a hapten-linker-large group conjugate of any one of claims 1 to 20.

35. A rapid assay kit of claim 34, wherein the kit further includes an indicator.

36. A rapid assay kit of claim 35, wherein the indicator is bound to the hapten-linker-large group conjugate.

37. A rapid assay kit of claim 35, wherein the indicator is bound to the ligand.

38. A rapid assay kit of any one of claims 34 to 37, which is a flow over kit.

39. A rapid assay kit of claim 38, which is a test strip.

40. A rapid assay kit of any one of claims 34 to 37, which is a flow through kit.

41. A process for binding a hapten-linker-large group conjugate of any one of claims 1 to 20 to a ligand comprising the steps of contacting the conjugate with a ligand capable of binding the hapten in the conjugate for a predetermined time where the reaction does not approach equilibrium.

42. The process according to claim 41, wherein the ligand is immobilised.

43. The process according to claim 41 or claim 42, wherein the ligand is contacted with a hapten before being contacted by the hapten-linker-large group conjugate.

44. The process according to claim 41 or claim 42, wherein the ligand is contacted with the hapten-linker-large group conjugate before or simultaneously with being contacted by a hapten.

45. A hapten-linker-large group conjugate of any one of the preceding claims, wherein the ligand is an immunoglobulin molecule.

46. A process for producing a hapten-linker-large group conjugate of any one of claims 1 to 20 is provided, including at least the steps of:
g) mixing an activated steroid hapten dissolved in an polar organic solvent with an aqueous solution comprising 1-10 molar equivalents of a heterobifunctional water-soluble linker;
h) allow the mixture to react; and
i) attach a large group to the remaining free functional linker group of the reaction hapten-linker product of step b).

47. The process according to claim 46, which includes an isolation step between steps b) and c) for isolating the hapten-linker product.

48. The process according to claim 46 or claim 47, wherein the final mixture has an aqueous content of between 2 and 30%.

49. The process according to any one of claims 46 to 48, wherein the final mixture has an aqueous content of between 5 and 15%.

50. The process according to any one of claims 46 to 49, wherein the final mixture has an aqueous content of about 10%.

51. The process according to any one of claims 46 to 50, wherein the step b) reaction time is in the order of up to 24 hours.

52. The process according to any one of claims 46 to 51, wherein the reaction in b) takes place at room temperature.

53. The process according to any one of claims 46 to 52, wherein the reaction in b) takes place at substantially neutral pH.

54. The process according to any one of claims 46 to 53, wherein the aqueous solution of step a) comprises 2-5 molar equivalents of a heterobifunctional water-soluble linker when compared with the activated steroid.

55. The process according to any one of claims 46 to 54, wherein the activated steroid is an activated ester of a steroid.

56. The process according to any one of claims 46 to 55, wherein the activated steroid is a succinimide ester of a steroid.

57. The process according to any one of claims 46 to 56, wherein the heterobifunctional linker is carboxyl at one end and amino at the other end.

58. The process according to any one of claims 46 to 57, wherein the polar organic solvent is selected from the group comprising DMF, DMSO, acetone and THF.
